(19) United States
(12) Patent Application Publication
(10) Pub. No.: US 2012/0239004 A1
(43) Pub. Date: Sep. 20, 2012

(54) CATHETER WITH FRICTION REDUCING SURFACE SHAPING

(75) Inventor: Paxton Wong, Maroubra, NSW (AU)

(73) Assignee: MK INTERNATIONAL PTY LTD, Maroubra, NSW (AU)

(21) Appl. No.: 13/502,999

(22) PCT Filed: Mar. 3, 2009
(86) PCT No.: PCT/AU09/00246

§ 371 Date: Apr. 19, 2012

(30) Foreign Application Priority Data
Mar. 3, 2008 (AU) 2008901027

(51) Int. Cl. A61M 27/00

(52) U.S. Cl. 604/540

(57) ABSTRACT

A catheter comprising a tube member having an open and a closed end (7) and either a head (8) at the tube member closed end, having a diameter greater than the tube outer diameter and/or a circumferential neck (12) adjacent the tube member closed end, the neck outer diameter being less than the tube outer diameter and greater than the tube outer diameter. In alternative embodiments, the closed tube end my have one or more dimples disposed on the tube surface, of a depth less than the tube wall thickness; one or more projections disposed on the tube surface; or more flute portions disposed on the tube surface, of a depth less than the tube wall thickness. One or more fluid apertures (9, 10) are disposed in the tube member, in or adjacent the closed end, neck, dimples, projections or fluted portions.

### FIELD OF THE INVENTION

The present invention relates to catheters.

The invention has been developed primarily with respect to disposable or single use urinary catheters and will be described hereinafter with reference to this application. However, it will be appreciated that the invention is not limited to this particular field of use.

### BACKGROUND ART

Urinary catheters have long been known. The urinary catheter is a tube which is inserted into the urethra of a person to drain urine. These are most typically used during surgical and clinical procedures, or by those suffering serious spinal injuries.

Typical urinary catheters comprise a first end configured for receiving urinary fluid when inserted into a urethra and a second end configured to be connected to a bag or other receptacle or container to collect or drain urine. The first end of the catheter can be cannular or syringe shaped where the catheter tube is simply sliced at an angle to its cross-section. Also well known are catheters having a first end which is closed and in which two or more urinary fluid apertures, or eyelets, are disposed in the tube adjacent the closed end. The apertures are typically longitudinally spaced along the catheter tube and are radially offset.

Whilst catheters are often used in sterile or clinical environments, they can be used in sealed sterile field urinary catheter kits. One example of such a urinary catheter kit was invented by Dr. George O'Neil and is the subject of U.S. Pat. No. 4,652,259. These catheter kits are provided having a lubricated catheter tube disposed within the sterile urine collection bag and dispensable through an insertion tip or other opening for insertion into a urethra. Whilst catheter developments such as these employing an insertion tip have reduced the rate of infection in catheter use, conventional catheter tubes are widely used.

### GENESIS OF THE INVENTION

It is the genesis of the present invention to provide a catheter having reduced resistance to insertion and/or removal, or to provide a useful alternative.

### SUMMARY OF THE INVENTION

According to a first aspect of the present invention there is provided a catheter comprising:
a tube member having an outer diameter and an inner diameter and extending between an open end and a closed end;
a catheter head disposed at said tube member closed end and having a diameter greater than said tube member outer diameter and less than three times said tube member outer diameter; and
one or more fluid apertures disposed in said tube member, in said catheter head, or in said closed end.

According to a second aspect of the invention there is provided a catheter comprising:
a tube member having an outer diameter and an inner diameter and extending between an open end and a closed end;
a catheter neck circumferentially disposed about said tube member adjacent said tube member closed end, said neck having an outer diameter less than said tube member outer diameter and greater than said tube member inner diameter; and
one or more fluid apertures disposed in said tube member, in said neck, or in or adjacent said closed end.

According to a third aspect of the invention there is provided a catheter comprising:
a tube member having an outer diameter and an inner diameter and extending between an open end and a closed end;
a catheter neck circumferentially disposed about said tube member adjacent said tube member closed end, said neck having an outer diameter greater than said tube member outer diameter and less than three times said tube member outer diameter; and
one or more fluid apertures disposed in said tube member, in said neck, or in or adjacent said closed end.

According to a fourth aspect of the invention there is provided a catheter comprising:
a tube member having an outer diameter and an inner diameter and extending between an open end and a closed end;
one or more catheter dimples disposed in said tube member, said dimples extending from said tube member outer diameter towards said tube inner diameter a predetermined depth being less than a distance between said tube inner and our diameters; and
one or more fluid apertures disposed in said tube member, in or adjacent said one or more dimples, or in or adjacent said tube member closed end.

According to a fifth aspect of the invention there is provided a catheter comprising:
a tube member having an outer diameter and an inner diameter and extending between an open end and a closed end;
one or more catheter projections disposed on said tube member outside diameter, said projections extending outwardly from said tube member outer diameter a predetermined distance; and
one or more fluid apertures disposed in said tube member, in or adjacent said one or more catheter projections, or in or adjacent said tube member closed end.

According to another aspect of the invention there is provided a catheter comprising:
a tube member having an outer diameter and an inner diameter and extending between an open end and a closed end;
one or more fluted portions disposed in said tube member outer diameter, said fluted portions extending a depth less than a distance between said tube member inner and outer diameters; and
one or more fluid apertures disposed in said tube member, in or adjacent said fluted portions, or in or adjacent said closed end.

It can therefore be seen that there is advantageously provided a catheter tube which has one or more neck portions which act to break the surface tension of fluid in the urethra during insertion and/or removal. There is also advantageously provided a catheter having a catheter head with an outer diameter greater than the catheter tube which advantageously acts to reduce the resistance of the catheter during insertion and/or removal.

### BRIEF DESCRIPTION OF THE DRAWINGS

Preferred embodiments of the invention will now be described, by way of example only, with reference to the accompanying drawings.

FIG. 1 is a schematic cross-sectional side view of a catheter according to a first preferred embodiment;

FIG. 2 is a schematic cross-sectional side view of a catheter according to another preferred embodiment;

FIG. 3 is a schematic cross-sectional side view of a catheter according to another preferred embodiment;

FIG. 4 is a schematic cross-sectional side view of a catheter according to another preferred embodiment;

FIG. 5 is a schematic cross-sectional side view of a catheter according to another preferred embodiment;

FIG. 6 is a schematic cross-sectional side view of a catheter according to another preferred embodiment;

FIG. 7 is a schematic cross-sectional side view of a catheter according to another preferred embodiment;

FIG. 8 is a schematic cross-sectional side view of a catheter according to another preferred embodiment;

FIG. 9 is schematic a cross-sectional side view of a catheter according to another preferred embodiment; and

FIG. 10 is a schematic cross-sectional side view of a catheter according to another preferred embodiment;

FIG. 11 is a schematic cross-sectional side view of a catheter according to another preferred embodiment; and

FIG. 12 is a schematic cross-sectional side view of a catheter according to another preferred embodiment.

### DETAILED DESCRIPTION

It will be appreciated that throughout the description of the preferred embodiments that like reference numerals have been used to denote like components.

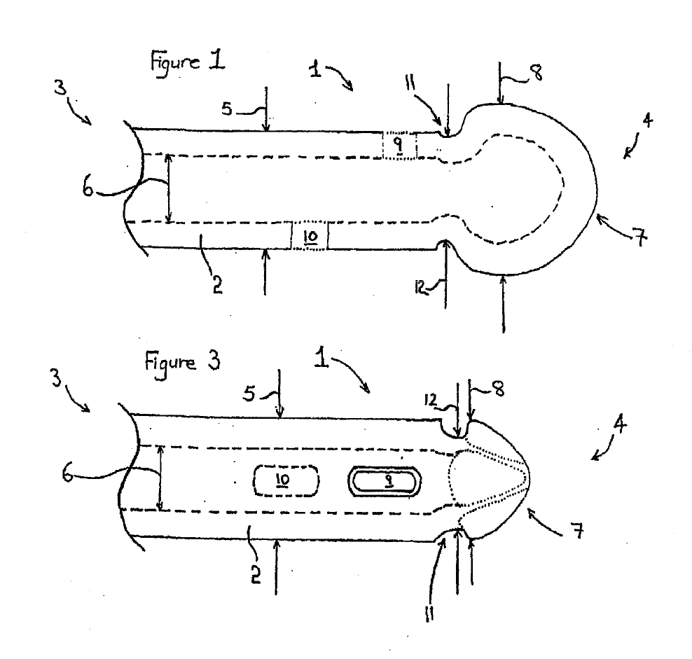

Referring to FIG. 1, there is shown a schematic cross-sectional side view of a catheter 1 according to the first preferred embodiment. The catheter 1 is formed from a tube member 2 which extends between an open end 3 and a closed end 4.

The tube member 2 has an outer diameter 5 in the range of 2 mm to 20 mm. The tube member 2 has an inner diameter 6 of between 1 mm and 15 mm. In the embodiment shown, the catheter length extending between open end 3 and closed end 4 is between 200 mm to 600 mm.

The catheter 1 includes a catheter head 7 disposed at the closed end 4. The catheter head 7 has a diameter 8 corresponding to the maximum diameter of the catheter head 7. The catheter 8 has a diameter greater than the outer diameter 5 of the tube member 2 but not greater than three times the outer diameter 5 of the tube 2. In the preferred embodiment of FIG. 1, the outer diameter 8 of the catheter head 1 is about 1½ times the outer diameter 5 of the tube member 2.

In the preferred embodiment of FIG. 1, the catheter head 7 is a bulbous shape and the catheter head diameter 8 described above refers to the maximum diameter thereof. On a portion of the catheter head 7 distal the tube member closed end 4 where the catheter head diameter 8 decreases from a maximum down to the tube member outer diameter 5, there is disposed a fluid aperture 9. A second fluid aperture 10 is disposed longitudinally further along the catheter tube.

A neck 11 is disposed at a base of the catheter head 7. The neck 11 has an outer diameter 12 which is less than the catheter head diameter 8 and the tube outer diameter 5.

In use, the closed end 4 of the catheter tube 2 is inserted into the urethra of a person. Once the catheter is inserted a predetermined distance, it remains for a predetermined period of time and any urine released by the bladder is collected by the fluid apertures 9 and 10 and drained through the inner diameter 6 of the tube member 2 out the open end 3 into a collection reservoir (not illustrated).

It will be appreciated that upon insertion, the catheter head 7 having a diameter greater than the tube member outer diameter 5 has the advantageous effect of reducing the resistance, particularly to insertion, faced by the catheter 1. It will be appreciated, however, that any preferred shaped catheter head can be provided if it has a maximum diameter being greater than the outer diameter 5 of the tube member 2 but less than three times that diameter.

It will also be appreciated that the catheter 1 can be formed from any preferred material such as silicone and the closed end 4 is most preferably formed by radio frequency cutting and sealing.

Further, it will also be appreciated that the fluid apertures 9 and 10 can be disposed in the tube member 2, as shown in FIG. 1, or may be disposed in or adjacent the closed end 4 or the catheter head, or in or adjacent the catheter neck 11. The location of the fluid apertures 9 and 10 can be any preferred.

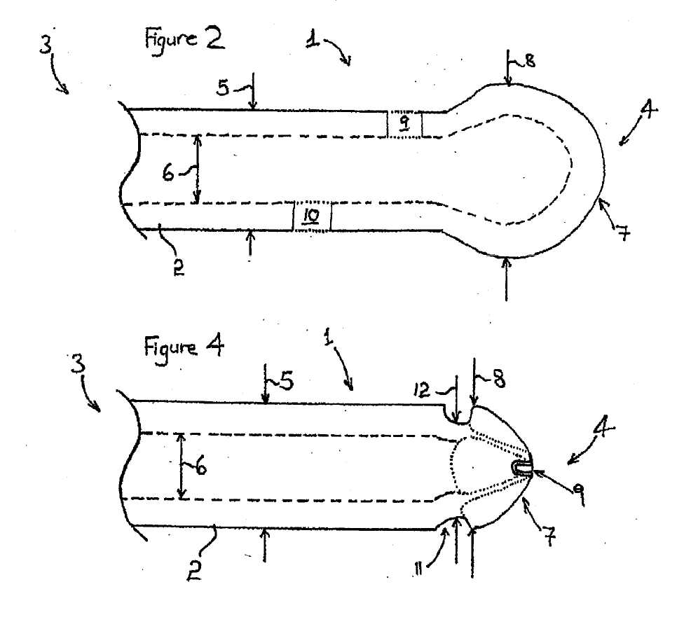

Turning to FIG. 2, there is shown a schematic cross-sectional side view of a catheter 1 according to another preferred embodiment. This embodiment is similar to that of FIG. 1 except that the neck 11 is removed and the catheter head 7 is disposed directly to the tube member 2. It will be appreciated that any preferred transition configuration can be provided at or intermediate the tube member 2 and the catheter head 7.

Referring to FIG. 3, there is shown a schematic cross-sectional side view of a catheter 1 according to another preferred embodiment of the invention. The catheter of this embodiment includes a tube member 2 extending between an open end 3 and closed end 4. The tube has an outer diameter 5 and an inner diameter 6. The tube member 2 includes a catheter neck 11 circumferentially disposed in the tube member 2 adjacent the closed end 4. The neck 11 has an outer diameter 12 which is less than the outer diameter 5 of the tube member 2 and greater than the inner diameter 6 of the tube member 2.

A catheter head 7 is formed intermediate the neck 11 and the closed end 4. In the embodiment shown, the head 7 has diameter (maximum diameter) 8 which is substantially the same diameter as the outer diameter 5 of the tube member 2.

In the preferred embodiment of the catheter 1 of FIG. 3, there is provided a pair of fluid apertures 9 and 10 configured to drain urinary fluid. The fluid aperture 9 closest the closed end 4 is disposed in the head 7 adjacent the neck 11. The second fluid aperture 10 is disposed longitudinally further down the tube member 2. However, it will be appreciated that the fluid apertures can be disposed in any preferred location in the tube member 2 intermediate the open end 3 and the closed end 4.

It can be seen that insertion of the catheter 1 into the urethra also advantageously reduces the resistance. This is due to the presence of the neck 11 disposed adjacent the closed end 4.

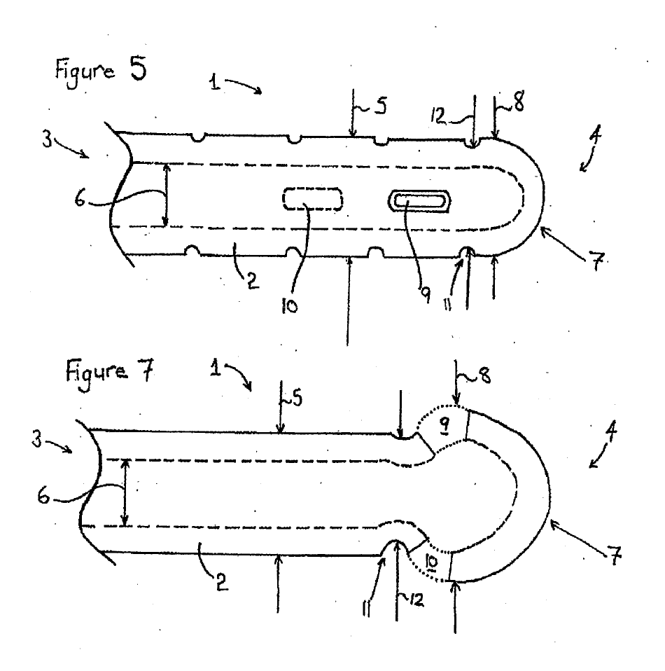

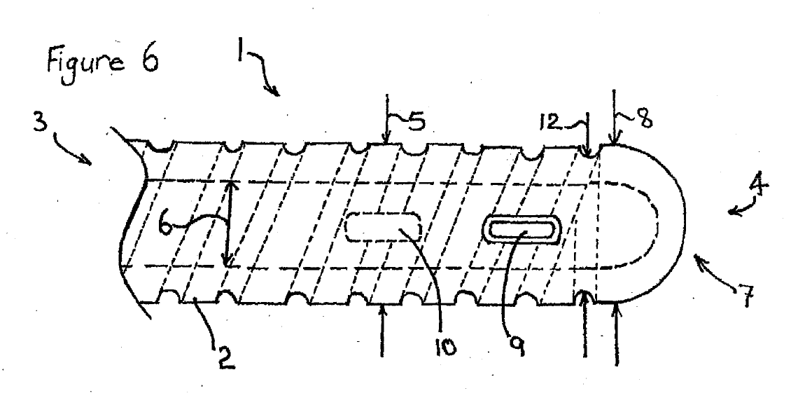

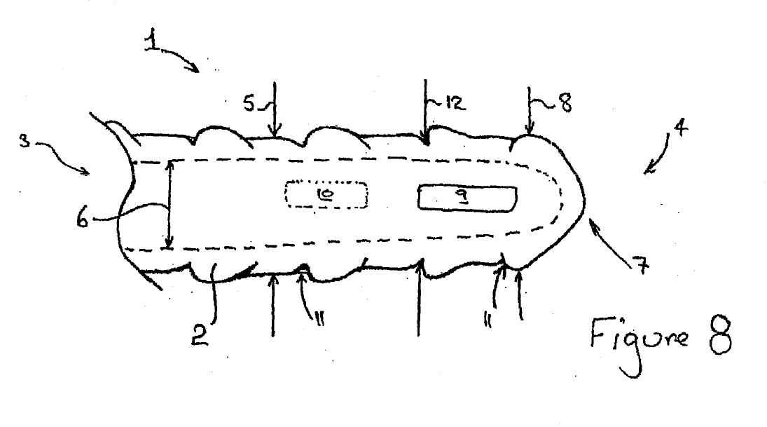

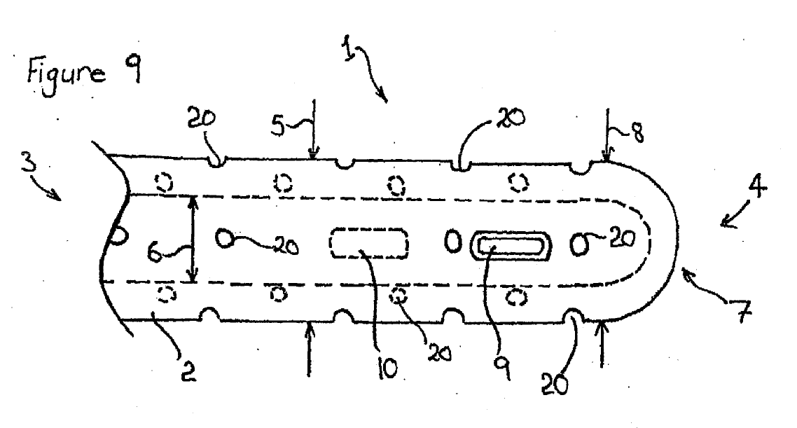

FIG. 4 is a schematic cross-sectional side view of a catheter 1 according to another preferred embodiment. This embodiment is similar to the catheter 1 of FIG. 3 where the fluid aperture 10 has been removed and fluid aperture 9 is disposed in the closed end 4. It will be appreciated that the fluid aperture can be disposed in any preferred location in the tube member 2, in or adjacent the catheter head 7 or in the closed end 4. Further, it will be appreciated that any number of fluid apertures can be used.

Referring now to FIG. 5, there is shown a schematic cross-sectional side view of a catheter 1 according to another preferred embodiment. In this embodiment, the catheter 1 includes a catheter tube 2 extending between an open end 3 and a closed end 4. As with the other embodiments of the invention above, the catheter tube 2 includes an outer diameter 5 and an inner diameter 6.

The catheter 1 includes a plurality of necks 11 having an outer diameter less than the outer diameter 5 of the tube member 2 but greater than the inner diameter 6 of the tube member 2. The necks 11 are circumferentially disposed about the tube member 2 and are spaced apart from each other longitudinally along the tube member 2 from adjacent the closed end 4. Although the fluid aperture 9 in the embodiment of FIG. 5 as shown intermediate a first neck 11 and the closed end 4, it will be appreciated that it can be disposed in any neck 11 or at another position along the length of the tube member 2 intermediate the open end 3 and closed end 4.

Although not illustrated, it will be appreciated that any one or more of the necks 11 shown in FIG. 5 can be discontinuous in that they do not extend continuously or uniformly all the way around the tube member 2. Further, it will be appreciated one or more of the necks 11 can be formed intermittently around the tube member 2 whereby the neck 11 includes a plurality of neck components interspersed with the tube member 2 being portions without a neck. That is, the neck 11 can be ‘dotted’ about the tube member 2.

Turning to FIG. 6, there is shown a schematic cross-sectional side view of a catheter according to another preferred embodiment. In this embodiment, the catheter 1 includes a tube member 2 extending between an open end 3 and a closed end 4. The tube member 2 includes an outer diameter 5 and an inner diameter 6. The catheter 1 includes a single neck 11 having an outer diameter less than the tube member outer diameter 5 and greater than the tube member inner diameter 6.

The neck 11 of the catheter 1 of this embodiment is helicly disposed about the tube member 2 and extends along a predetermined length along the tube member 2. The helical neck 11 extends from or adjacent from the head 8 along the tube member 2.

In an alternative embodiment (not illustrated) to that shown in FIG. 6, it will be appreciated that the neck 11 of this embodiment can be discontinuous similarly to that described with reference to the embodiment of FIG. 5. The helical neck 11 can be interspersed with the tube member 2 being portions in place of portions of the neck 11.

Referring to FIG. 7, there is shown a schematic cross-sectional side view of a catheter 1 according to another preferred embodiment. In this embodiment, the tube member 2 includes a neck 11 disposed adjacent the closed end 4. Intermediate the neck 11 and the closed end 4 there is disposed a catheter head 7 having a maximum diameter being larger than the outer diameter 5 of the tube member 2 but less than three times the outer diameter 5. In this embodiment, it will be appreciated that the catheter head 7 and the neck 11 disposed adjacent the head 7 at end distal the closed end advantageously reduces the resistance when inserting and removing the catheter from a urethra.

In the embodiment of FIG. 7, the fluid apertures 9 and 10 are disposed in the tube 2 at the catheter head 7 at a point where the catheter head 7 is distal from the closed end 4 and is adjacent the neck 11. In this way, the apertures 9 and 10 are disposed in the catheter head where it has a diameter less than its maximum diameter 8 so as to partially or totally face away from the closed end 4. This advantageously reduces any risk of tearing of the urethra by the tube member 2 about the aperture circumference in addition to reducing the resistance to insertion of the catheter.

It will be appreciated that the tube member 2 of the catheter 1 can have an outer diameter 5 which is uniform or which is non-constant (not illustrated). Likewise, it will be appreciated that the inner diameter 6 of the tube member 2 can also be uniform or it may be non-constant and vary (not illustrated). Furthermore, it will be appreciated that the catheter 1 of any one of the preferred embodiments can be used in a sterile field urinary catheter kit.

It will also be appreciated that in the embodiment of FIG. 1, for example, the head diameter 8 being wider than the outer diameter 5 of the tube member 2 can be used to break or rupture or a seal formed by a silicone tip at the exit to sterile field urinary catheter reservoir. Yet further, it will be appreciated that any preferred lubricants can be used with the catheter 1 of the preferred embodiments including, but not limited to, gels and hydrophilic salts.

Referring to FIG. 8, there is shown a schematic cross-sectional side view of another preferred embodiment of a catheter 1. In this embodiment, the catheter 1 includes a tube member 2 which extends between an open end 3 and a closed end 4.

The tube member 2 has an outer diameter 5 in the range of 2 mm to 20 mm. As with the preferred embodiment described above, the tube member 2 has an inner diameter 6 of between 1 mm and 15 mm. The catheter 1 extends a length of between 200 mm to 600 mm between the open end 3 and closed end 4.

The tube member 2 includes a catheter neck 11 circumferentially disposed about the tube member 2 adjacent the closed end 4. The catheter neck 11 has an outer diameter which is greater than the outer diameter of the tube member 2 and, as shown in the preferred embodiment, is about 1.25 times the tube member outer diameter 6. The catheter 1 further includes a pair of fluid apertures 9 and 10 configured to drain urinary fluid and the apertures 9 and 10 can be disposed at any preferred location along the length of the tube member 2.

The catheter neck 11 extends along the tube member 2 a length of between 1 mm and 20 mm. Upon insertion of the catheter 1 into a urethra this embodiment has the advantageous effect of reducing the resistance faced by the catheter 1. It will be appreciated, however, that any preferred shaped catheter neck 11 can be provided if it has a maximum diameter being greater than the outer diameter 5 of the tube member 2 but less than three times that diameter.

It will be appreciated that in other embodiments of the invention of FIG. 8, not illustrated, the catheter 1 can include a plurality of catheter necks longitudinally spaced apart along the tube member 2. Furthermore, it will be appreciated that the inner 6 and/or outer 5 diameters of the tube member 2 can be constant or can vary as desired.

Referring to FIG. 9, there is shown a schematic cross-sectional side view of a catheter 1 according to another preferred embodiment. The catheter 1 includes a tube member 2 having an outer diameter 5 and an inner diameter 6. The catheter 1 extends between an open end 4 and a closed end 3. A plurality of catheter dimples 20 are disposed in the tube member 2. The dimples 20 extend from the tube member outer diameter 5 towards the tube inner diameter 5 a predetermined depth being less than a distance between said tube inner and our diameters.

The dimples 20 are disposed in the tube member 2 from adjacent the closed end 4 and the tube member 2 is dimpled therefrom along a predetermined length towards the open end 3. The catheter 1 includes two fluid apertures 9 and 10 disposed in said tube member but it will be appreciated they can be disposed in or adjacent said one or more dimples, or in or adjacent said tube member closed end. The presence of the dimples advantageously acts to minimise the resistance to insertion and removal of the catheter 1 from a urethra.

In another preferred embodiment (not illustrated), the dimples 20 can be substituted with projections extending from the tube member 2 a distance not greater than twice the outer diameter 5. This advantageously also acts to minimise the resistance to insertion and removal of the catheter 1 from a urethra.

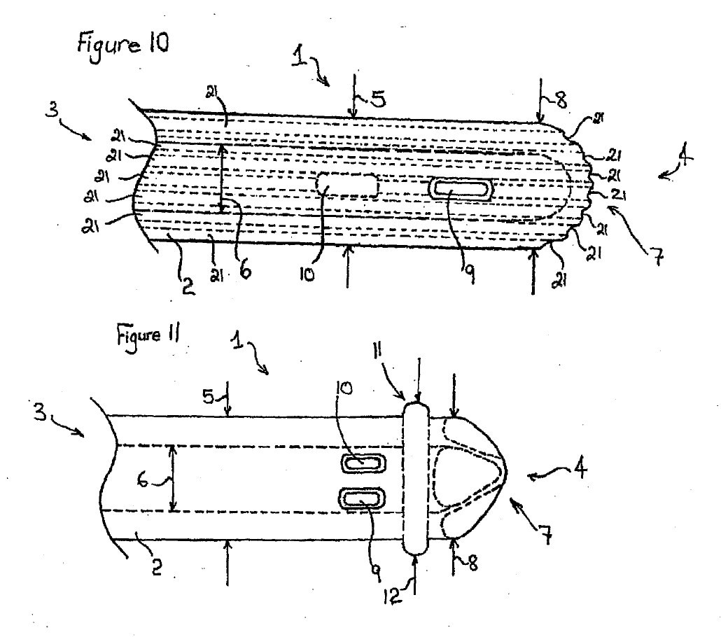

Referring to FIG. 10, there is shown a schematic cross-sectional side view of a catheter 1 according to another preferred embodiment. In this embodiment, the catheter 1 includes a tube member 2 having an outer diameter 5 and an inner diameter 6. The tube member 2 extends between an open end 3 and a closed end 4. A plurality of fluted portions 21 are disposed in the tube member outer diameter 5. The fluted portions 21 extend a depth less than a distance between the tube member inner 6 and outer 7 diameters.

The catheter 1 includes a plurality of fluid apertures 9 and 10 disposed in the tube member 2. However, it will be appreciated the fluid apertures can be disposed in any desired location in order to drain urine such as in or adjacent the fluted portions, or in or adjacent the closed end 4. It can be seen the fluted portions 21 advantageously act to minimise the resistance to insertion and removal of the catheter 1 from a urethra.

Turning to FIG. 11, there is shown a schematic cross-sectional side view of a catheter 1 according to another preferred embodiment. The catheter 1 of this embodiment includes a tube member 2 extending between an open end 3 and closed end 4. The tube has an outer diameter 5 and an inner diameter 6.

The tube member 2 includes a catheter neck 11 circumferentially disposed about the tube member 2 adjacent the closed end 4. The neck 11 has an outer diameter 12 which is about 1.25 times greater than the outer diameter 5 of the tube member 2. Preferably, the outer diameter 12 of the neck 11 is not greater than three times the outer diameter 5 of the tube member 2.

A catheter head 7 is formed intermediate the neck 11 and the closed end 4. In the embodiment shown, the head 7 has diameter (maximum diameter) 8 which is substantially the same or less in diameter than the outer diameter 5 of the tube member 2.

There is also provided a pair of fluid apertures 9 and 10 configured to drain urinary fluid. The fluid apertures 9 and 10 are spaced apart along the tube 2 about the same distance from the neck 11 and they are circumferentially offset. However, it will be appreciated that the fluid apertures can be disposed in any preferred location in the tube member 2 intermediate the open end 3 and the closed end 4.

It can be seen that insertion of the catheter 1 into the urethra also advantageously reduces the resistance. This is due to the presence of the neck 11 disposed adjacent the closed end 4. Although not illustrated, it will be appreciated that a plurality of longitudinally spaced apart necks 11 can be disposed on the tube member 2. Further, the necks 11 can be discontinuous or non-uniform in maximum diameter 12.

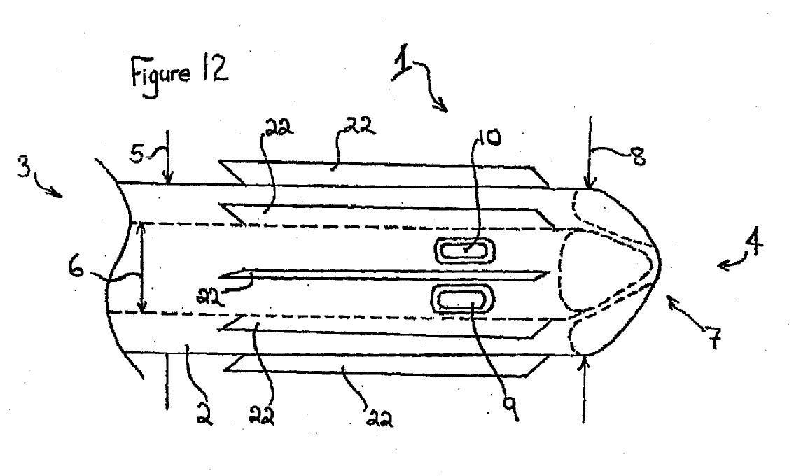

Referring to FIG. 12, there is shown a schematic cross-sectional side view of a catheter 1 according to another preferred embodiment. The catheter 1 includes a tube member 2 having an outer diameter 5 and an inner diameter 6. The tube member 2 extends between an open end 3 and a closed end 4. A plurality of catheter projections 22 are disposed on the tube member outside diameter 5. The projections 22 extending outwardly from said tube member outer diameter 5 a predetermined distance. The catheter 1 further includes a pair of fluid apertures 9 and 10 disposed in the tube member 2. It will be appreciated that the fluid apertures 9 and 10 can be disposed in any preferred location such as in or adjacent the one or more catheter projections 22, or in or adjacent the tube member closed end 4.

The projections 22 are spaced apart on the tube member outer diameter 5 and extend a predetermined length along said tube member from adjacent the closed end 4. The projections 22 extend outwardly a distance less than two times the tube member outer diameter 5.

It can be seen the projections 22 advantageously act to minimise the resistance to insertion and removal of the catheter 1 from a urethra. It will be appreciated that the projections 22 can longitudinally extending along the tube member 2 any desired length and can be discontinuous or ‘dotted’ therealong. Further, ribbed projections or the like may also be used.

Yet further, it will be appreciated that the neck 11 can include one or more portions which have a diameter equal to the outer diameter 6 of the tube member 2. In this way, a circumferentially discontinuous or non-uniform neck 11 is formed. Similarly in the case of the preferred embodiments described above, the head 7 or neck 11 can include one or more portions having an outer diameter that is equal to the outer diameter 5 of the tube member 2. For example, the head 7 or neck 11 can include one or more longitudinally extending portions having a diameter substantially equal to the tube member 2 outer diameter 6.

The foregoing describes only preferred embodiments of the present invention and modifications, obvious to those skilled in the art, can be made thereto without departing from the scope of the present invention.

The term “comprising” (and its grammatical variations) as used herein is used in the inclusive sense of “including” or “having” and not in the exclusive sense of “consisting only of”.

1. A catheter comprising:
a tube member having an outer diameter and an inner diameter and extending between an open end and a closed end;
a catheter head disposed at said tube member closed end and having a diameter greater than said tube member outer diameter and less than three times said tube member outer diameter; and
one or more fluid apertures disposed in said tube member, in said catheter head, or in said closed end.

2. A catheter according to claim 1 wherein said catheter head diameter is twice said tube member outer diameter.

3. A catheter according to claim 1 wherein said catheter head is bulbous.

4. A catheter according to claim 1 comprising a neck portion disposed at a base of said catheter head.

5. A catheter according to claim 4 wherein said neck diameter is less than said tube member outer diameter.

6. A catheter according to claim 4 wherein said neck outer diameter is greater than one-third of said tube member outer diameter and less than said tube member outer diameter, or is greater than said tube member inner diameter and less than said tube member outer diameter.

7. A catheter according to claim 1 wherein said tube member outer diameter and/or said tube member inner diameter are non constant.

8. A catheter according to claim 1 wherein said tube member extends between 200 mm to 600 mm in length between said open and closed ends of said tube member, said tube member inner diameter between 2 mm to 10 mm, and said tube member outer diameter between 3 mm to 20 mm.

9. A catheter comprising:
a tube member having an outer diameter and an inner diameter and extending between an open end and a closed end;
a catheter neck circumferentially disposed about said tube member adjacent said tube member closed end, said neck having an outer diameter less than said tube member outer diameter and greater than said tube member inner diameter; and
one or more fluid apertures disposed in said tube member, in said neck, or in or adjacent said closed end.

10. A catheter according to claim 9 comprising a catheter head formed intermediate said neck and said tube member closed end, said head outer diameter being greater than said tube member outer diameter.

11. A catheter according to claim 9 wherein an outer diameter of said neck is less than said tube member outer diameter and greater than one-third of said tube member outer diameter.

12. A catheter according to claim 10 wherein part or all of said one or more fluid apertures is disposed on said catheter head adjacent said neck portion where said catheter head has an outer diameter less than a maximum catheter head outer diameter.

13. A catheter according to claim 9 including a plurality of catheter necks disposed in said tube member longitudinally spaced apart along said tube member from said closed end.

14. A catheter according to claim 9 wherein said neck is helicly disposed about said tube member and extends a predetermined distance therealong.

15. A catheter according to claim 9 wherein said neck is discontinuous or intermittently formed.

16. A catheter according to claim 9 wherein said tube member extends between 200 mm to 600 mm in length between said open and closed ends of said tube member, said tube member inner diameter is between 2 mm to 10 mm, and said tube member outer diameter is between 3 mm to 20 mm.

17. A catheter comprising:
a tube member having an outer diameter and an inner diameter and extending between an open end and a closed end;
a catheter neck circumferentially disposed about said tube member adjacent said tube member closed end, said neck having an outer diameter greater than said tube member outer diameter and less than three times said tube member outer diameter; and
one or more fluid apertures disposed in said tube member, in said neck, or in or adjacent said closed end.

18. A catheter according to claim 17 wherein said neck outer diameter is between 1.25 to 1.5 times said tube member outer diameter.

19. A catheter according to claim 17 wherein said neck extends along said tube member a length of between 1 mm and 20 mm.

20. A catheter according to claim 17 comprising a plurality of catheter necks longitudinally spaced apart along said tube member.

21. A catheter according to claim 17 wherein said tube member inner diameter and/or outer diameter is constant.

22. A catheter according to claim 17 wherein said tube member extends between 200 mm to 600 mm in length between said open and closed ends of said tube member, said tube member inner diameter between 2 mm to 10 mm, and said tube member outer diameter between 3 mm and 20 mm.

23. A catheter according to claim 17 wherein said neck is helicly disposed about said tube member and extends a predetermined distance therealong.

24. A catheter according to claim 17 wherein said neck is discontinuous or intermittently formed.

25. A catheter comprising:
a tube member having an outer diameter and an inner diameter and extending between an open end and a closed end;
one or more catheter dimples disposed in said tube member, said dimples extending from said tube member outer diameter towards said tube inner diameter a predetermined depth being less than a distance between said tube inner and our diameters; and
one or more fluid apertures disposed in said tube member, in or adjacent said one or more dimples, or in or adjacent said tube member closed end.

26. A catheter comprising:
a tube member having an outer diameter and an inner diameter and extending between an open end and a closed end;
one or more catheter projections disposed on said tube member outside diameter, said projections extending outwardly from said tube member outer diameter a predetermined distance; and
one or more fluid apertures disposed in said tube member, in or adjacent said one or more catheter projections, or in or adjacent said tube member closed end.

27. A catheter according to claim 32 comprising a plurality of substantially parallel projections spaced apart about said tube member outer diameter, said catheter projections extending a predetermined length along said tube member and extending outwardly a distance less than two times said tube member outer diameter.

28. A catheter comprising:
a tube member having an outer diameter and an inner diameter and extending between an open end and a closed end;
one or more fluted portions disposed in said tube member outer diameter, said fluted portions extending a depth less than a distance between said tube member inner and outer diameters; and
one or more fluid apertures disposed in said tube member, in or adjacent said fluted portions, or in or adjacent said closed end.
